(19) United States
(12) Patent Application Publication
(10) Pub. No.: US 2015/0182449 A1
(43) Pub. Date: Jul. 2, 2015

(54) TRADITIONAL CHINESE MEDICINE COMPOSITION FOR PIGMENT REPAIR

(71) Applicant: Yuan Yuan, Beijing (CN)

(72) Inventor: Yuan Yuan, Beijing (CN)

(73) Assignee: Shang Chen Mei Bang (Beijing) Biotechnology Co., Ltd., Beijing (CN)

(21) Appl. No.: 14/144,561

(22) Filed: Dec. 31, 2013

(51) Int. Cl. A61K 8/98; A61Q 19/02; A61K 8/73; A61K 8/97; A61K 8/67

(52) U.S. Cl. CPC A61K 8/981; A61K 8/97; A61K 8/676; A61K 8/73; A61Q 19/02

(57) ABSTRACT

The invention provides a composition for use in traditional Chinese medicine to repair the pigment, wherein said composition comprises the following parts by weight of the composition ratio of raw material: 5-10 parts water, Rhodiola extract 2-6 copies, snow Lotus root extract 3-5 parts, 1-5 parts Egeria motherwort extract, vitamin-C2-3 copies, copies of oat amino acids 2-5, 2-3 parts of dextran, 1-2 parts of emu oil, Astragalus extract 2-3 parts, 4-5 parts of ginseng root extract, angelica extract 2-3 parts. This composition helps dilute pigment and redness, thickening of the skin to make the skin more resistant to ease fragile, net flashed white luster.

### CROSS-REFERENCE TO RELATED APPLICATIONS

This application claims the priority benefit of Chinese Patent Application Serial No. 201310703312.4, filed on Dec. 19, 2013; Chinese Patent Application Serial No. 201310685138.5, filed on Dec. 13, 2013; Chinese Patent Application Serial No. 201310687223.5, filed on Dec. 13, 2013; Chinese Patent Application Serial No. 201310685115.4, filed on Dec. 13, 2013; Chinese Patent Application Serial No. 201310684837.8, filed on Dec. 13, 2013; Chinese Patent Application Serial No. 201310685112.0, filed on Dec. 13, 2013; Chinese Patent Application Serial No. 201310686861.5, filed on Dec. 13, 2013; Chinese Patent Application Serial No. 201310687218.4, filed on Dec. 13, 2013; Chinese Patent Application Serial No. 201310686865.3, filed on Dec. 13, 2013; Chinese Patent Application Serial No. 201310687250.2, filed on Dec. 13, 2013; Chinese Patent Application Serial No. 201310684846.7, filed on Dec. 13, 2013; Chinese Patent Application Serial No. 201310685166.7, filed on Dec. 13, 2013; and Chinese Patent Application Serial No. 201310687246.6, filed on Dec. 16, 2013, the disclosure of all of which are hereby incorporated by reference in their entirety.

### FIELD OF THE INVENTION

The invention belongs to the field of traditional Chinese medicine, in particular to a traditional Chinese medicine composition for pigment repair.

### BACKGROUND OF THE INVENTION

Human skin problems as large divided pigmentation, aging, inflammation and other phenomena, but there are also inextricably linked between them, such as skin color is mainly affected by melanin, and dry skin, aging; skin pocked; sun UV radiation can cause skin pigmentation, etc. are the main reason. The commonly used additives in cosmetics, chemical synthesis are: hydroquinone, retinoic acid, azelaic acid Kui, the effects were relatively simple, and most have some side effects, such as cytotoxicity, skin discoloration and so on. The protective effect of traditional Chinese medicine has multiple, fully inhibit melanin production.

### SUMMARY OF THE INVENTION

The present invention can help dilute the pigments and redness, thickening of the skin to make the skin more resistant to ease fragile, net flashed white luster.

Techniques of the present invention are: the kind of traditional Chinese medicine composition for pigment repair, wherein the ingredients: water 1-11, 2-12 Rhodiola extract, snow lotus root extract 3-13, fine 4-14 Motherwort leaf extract, vitamin-C5-15, oat amino acids 6-16, 7-17 dextran, emu oil 8-18, 9-19 astragalus extract, ginseng root extract 10-20, Angelica Extract 11-21.

Among them, the ingredients may be: water 1, 2 Rhodiola extract, snow lotus root extract 3, 4 Egeria motherwort extract, vitamin-C5, oat amino acids 6, dextran 7, 8 emu oil, 9 astragalus extract, ginseng root extract 10, Angelica Extract 11.

Among them, the ingredients may be: water 11, 12 Rhodiola extract, snow lotus root extract 13, 14 Egeria motherwort extract, vitamin-C15, oat amino acids 16, dextran 17, emu oil 18, 19 astragalus extract, ginseng root extract 20, Angelica Extract 21.

The components are extracted through conventional methods together with accessories made of Essence.

Also provides a composition for use of the medicine, sooner or later, after suitable cleanser, spray in the face and neck 2-3 times.

Pharmacological Effects:

Rhodiola (Rhodiola), a perennial herb, 10-20 cm. Root stout, conical, fleshy, brownish yellow, with numerous fibrous roots neck, Rhizome short, rough shape, cylindrical, are arranged most imbricate scaly leaves. From the top of the stem of the leaf axils draw several stems, fleshy leaves both top and bottom of the stems, leaves oval, serrated edge, apex acute, base cuneate, almost sessile. Cymes raw, red flowers. July-September harvest. Snow lotus, Tibetan, Tibet, Xinjiang, said just fruit Suba people in the region known as Carl Riley, Asteraceae perennial herb. Categories are cotton head snow lotus, snow lotus bud large, jellyfish snow lotus flowers with whole plant.

Egeria Sweet, spicy, bitter, cool. Blood, blood stasis, menstruation, elimination of water. Indications irregular menstruation, Tailou dystocia, less than afterbirth, postpartum blood dizzy, bleeding, abdominal pain, sore carbuncle.

Dextran, also known as dextran, in the presence of certain microorganisms in a growth process of secretion of mucus. Dextran having a higher molecular weight, with different types and growth conditions of the microorganisms, there are differences in the structure.

Astragalus, also known as cotton stilbene. Perennial herb, 50-100 cm. Astragalus medicinal than 2000 years of history, it has enhanced immune function, liver, diuretic, anti-aging, anti-stress, blood pressure and body wider antibacterial effect.

Ginseng root: root is fleshy with ginseng, used medicinally. Ginseng belongs to Araliaceae, grown mainly in East Asia, especially in cold regions.

Angelica, alias dry return, horsetail Angelica, which Qin, horsetail return, clouds return, West Angelica, Angelica Min, Kim Angelica, Angelica body, culvert final end, udo perennial herb in China located in Gansu, Yunnan, Sichuan, Qinghai, Shaanxi, Hunan, Hubei, Guizhou and other places around are cultivated. Angelica root can be used as medicine, is one of the most commonly used medicine.

After the composition of the present invention, the above ingredients, a synergistic effect of its efficacy.

The beneficial effects of the invention: Herbal extract composition of the invention inhibit melanoma cells, inhibition of tyrosinase, anti-free radical and UV effect, can effectively inhibit skin melanin production, with excellent whitening effect. The composition does not contain synthetic substances, toxic side effects, skin irritation, stable, safe and reliable.

### DETAILED DESCRIPTION OF EMBODIMENTS

### Example 1

A Chinese medicine for repairing pigment composition, wherein said composition comprises the following parts by weight of the composition ratio of raw materials: 5-6 parts water, 2-4 parts of Rhodiola extract, snow lotus root extract 3-4, 1-2 parts Egeria motherwort extract, vitamin-C2-3 copies, oat amino adds 2-3 parts, dextran 2-3 parts, 1-2 parts emu oil, astragalus extract 2-3 parts, 4-5 parts of ginseng root extract, angelica extract 2-3 parts.

### Example 2

A Chinese medicine composition for pigment repair, wherein said composition comprises the following parts by weight of the composition ratio of the raw materials: 7-8 parts water, Rhodiola extract 2-3 parts, snow lotus root extract 3-4, 4-5 parts Egeria motherwort extract, vitamin-C2-3 copies, oat amino adds 4-5 parts, dextran 2-3 parts, 1-2 parts emu oil, astragalus extract 2-3 parts, 4-5 parts of ginseng root extract, angelica extract 2-3 parts.

Usage: apply morning and evening after cleanser, spray in the face and neck 2-3 times.

The kinds of Chinese medicine composition for pigment repaired after three years of clinical tests, recording 200 cases, in order to significantly improve the skin condition as a standard, the total efficiency of 98%. With activation, whitening, super moisturizing protective function, can inhibit the formation of melanin and melanin matter Angelica and other plant extracts to restore the formation of melanin in the epidermis exclusion, dilute yellowish, make skin white and transparent.

The following referenced cases are herein incorporated: U.S. Nonprovisional patent application Ser. No. 14/______, filed Dec. 30, 2013 with Attorney Docket No. ShangChen-13-01, and entitled “A traditional Chinese medicine composition that has the effect of soothing and cleaning face”; U.S. Nonprovisional patent application Ser. No. 14/______, filed Dec. 30, 2013 with Attorney Docket No. ShangChen-13-02, and entitled “A Traditional Chinese medicine extract composition with whitening skin care function and its preparation method”; U.S. Nonprovisional patent application Ser. No. 14/______, filed Dec. 30, 2013 with Attorney Docket No. Shangchen-13-04, and entitled “A traditional Chinese medicine composition for repair of acne”; U.S. Nonprovisional patent application Ser. No. 14/______, filed Dec. 30, 2013 with Attorney Docket No. Shangchen-13-05, and entitled “A traditional Chinese medicine composition for anti-wrinkle”; U.S. Nonprovisional patent application Ser. No. 14/______, filed Dec. 30, 2013 with Attorney Docket No. Shangchen-13-06, and entitled “A traditional Chinese medicine composition for dispelling wrinkles and black eye and its preparation method”; U.S. Nonprovisional patent application Ser. No. 14/______, filed Dec. 30, 2013 with Attorney Docket No. Shangchen-13-07, and entitled “A Chinese medicine composition for nourishing body care and its preparation method”; U.S. Nonprovisional patent application Ser. No. 14/______, filed Dec. 30, 2013 with Attorney Docket No. Shangchen-13-08, and entitled “Chinese medicine composition that having acne cleansing effect”; U.S. Nonprovisional patent application Ser. No. 14/______, filed Dec. 30, 2013 with Attorney Docket No. Shangchen-13-09, and entitled “An anti-aging skin rejuvenation medicine composition and its preparation method”; U.S. Nonprovisional patent application Ser. No. 14/______, filed Dec. 30, 2013 with Attorney Docket No. Shangchen-13-10, and entitled “Chinese medicine composition for pigment desalination”; U.S. Nonprovisional patent application Ser. No. 14/______, filed Dec. 30, 2013 with Attorney Docket No. Shangchen-13-011, and entitled “Chinese medicine composition for acne scar repair”; U.S. Nonprovisional patent application Ser. No. 14/______, filed Dec. 30, 2013 with Attorney Docket No. Shangchen-13-012, and entitled “Chinese medicine composition for Prevent from aging and soothing the skin”; and U.S. Nonprovisional patent application Ser. No. 14/______, filed Dec. 30, 2013 with Attorney Docket No. Shangchen-13-013, and entitled “Chinese medicine composition for Nourishing the skin and its preparation method”, the disclosure of all of which are herein incorporated by reference in its entirety.

What is claimed is:

1. A traditional Chinese medicine composition for pigment repair, wherein said composition comprises the following parts by weight of the composition ratio of raw material: 5-10 parts water, 2-6 parts of Rhodiola extract, snow lotus root extract 3-5 parts, Egeria 1-5 parts motherwort extract, vitamin-C2-3 copies, copies of oat amino acids 2-5, 2-3 parts of dextran, 1-2 parts of emu oil, astragalus extract 2-3 parts matter, 4-5 parts of ginseng root extract, angelica extract 2-3 parts.

2. Where the Board composition according to claim 1, wherein the components are: 5-6 parts water, 2-4 parts of Rhodiola extract, snow lotus root extract 3-4, Egeria motherwort extract 1-2 parts of vitamin-C2-3 copies, oat amino acids 2-3 parts, dextran 2-3 parts, 1-2 parts of emu oil, 2-3 parts astragalus extract, ginseng root extract product 4-5 copies, Angelica Extract 2-3 parts.

3. Where the Board composition according to claim 1, wherein the components are: 7-8 parts water, Rhodiola extract 2-3 parts, snow lotus root extract 3-4, Egeria motherwort extract 4-5 parts of vitamin-C2-3 copies, copies of oat amino acids 4-5, 2-3 parts of dextran, 1-2 parts of emu oil, 2-3 parts astragalus extract, ginseng root extract product 4-5 copies, Angelica Extract 2-3 parts.

4. Chinese medicine composition as claimed in claim 1, wherein the components extracted through conventional methods together with accessories made of Essence.

5. The Essence of Chinese medicine made composition according to claim its use is suitable for the morning and evening after cleanser, spray in the face and neck 2-3 times.
